(19) United States
(12) Patent Application Publication
(10) Pub. No.: US 2012/0082650 A1
(43) Pub. Date: Apr. 5, 2012

(54) METHODS FOR TREATING PARKINSON'S DISEASE AND OTHER DISORDERS OF DOPAMINERGIC NEURONS OF THE BRAIN

(75) Inventors: Raymond T. Bartus, San Diego, CA (US); Joao Siffert, San Diego, CA (US)

(73) Assignee: Ceregene, Inc., San Diego, CA (US)

(21) Appl. No.: 13/078,897

(22) Filed: Apr. 1, 2011

Related U.S. Application Data
(60) Provisional application No. 61/320,654, filed on Apr. 2, 2010.

(51) Int. Cl. A61K 48/00; A61P 25/16; A61P 25/00; A61K 38/18

(52) U.S. Cl. 424/93.21; 514/44 R; 514/8.4

(57) ABSTRACT

A specific clinical protocol for use toward therapy of defective, diseased and damaged neurons in the mammalian brain, of particular usefulness for treatment of neurodegenerative conditions such as Parkinson's disease. The protocol is practiced by directly delivering a definite concentration of a nerve growth factor via delivery of the protein, an expression vector operably encoding the nerve growth factor, or grafting a donor cell containing such an expression vector into the substantia nigra and preferably also the striatum. The method stimulates growth of targeted neurons, and reversal of functional deficits associated with the neurodegenerative disease being treated.

### CROSS REFERENCE TO RELATED APPLICATION(S)

This application claims the benefit of priority under 35 U.S.C. §119(e) of U.S. Ser. No. 61/320,654, filed Apr. 2, 2010, the entire content of which is incorporated herein by reference in entirety.

### BACKGROUND OF THE INVENTION

1. Field of the Invention

The present invention relates generally to methods for treatment of neurodegenerative disease such as Parkinson's disease by administering therapeutic nerve growth factors into the mammalian brain.

1. Background Information

Parkinson's disease is a common neurodegenerative disorder characterized clinically by bradykinesia, rigidity, tremor, and gait dysfunction, and pathologically by degeneration of dopamine neurons in the substantia nigra pars compacta (substantia nigra) and by projection to the striatum (including the putamen). Present therapies provide satisfactory disease control for most patients, particularly in the early stages. However, no treatments protect against the continued degeneration of these neurons and, over time, all therapies fail.

For example, chronic levodopa treatment is associated with motor complications, does not control potentially disabling features such as falling and dementia, and fails to prevent disease progression. Olanow et al., Neurology 72:suppl 4:S1-S136 (2009). Thus, more effective treatments that improve clinical disease control and slow progression are urgently needed.

Neurotrophic factors might improve neuronal function and protect against neurodegeneration. Glial-cell-derived neurotrophic factor (GDNF) protects dopamine neurons in in-vitro and animal models of Parkinson's disease. Lin et al., Science 260:1130-1132 (1993) and Gash et al., Nature 380:252-255 (1996). Neurturin (NRTN) is a naturally occurring structural and functional analogue of GDNF (Kotzbauer et al., Nature 384:467-470 (1996)) that has been demonstrated to improve dopaminergic activity in aged monkeys (Herzog et al., Mov Disord 22:1124-1132 (2007)) and protect dopamine neurons in animal models of Parkinson's disease. Horger et al., J Neurosci 18:4929-4937 (1998); Gasmi et al., Neurobiol Dis 27:67-76 (2007); Gasmi et al., Mol Ther 15:62-68 (2007); and Kordower et al., Ann Neurol 60:706-715 (2006).

However, the use of neurotrophic factors as a treatment for neurodegenerative diseases has proven extremely difficult, in large part due to obstacles that preclude targeted delivery of adequate and sustained levels of protein to the degenerating neurons. Results from subsequent open-label trials have shown benefits of continuous infusion of GDNF into the putamen in patients with advanced Parkinson's disease. Gill et al., Nat Med 9:589-595 (2003) and Slevin et al., J Neurosurg 102:216-222 (2005). However, these results were not confirmed in double-blind studies, (Nutt et al., Neurology 60:69-73 (2003) and Lang et al., Ann Neurol 59:459-466 (2006)) possibly because the trophic factor was not adequately distributed throughout the target region. Kordower et al., Ann Neurol 46:419-424 (1999) and Salvatore et al., Exp Neurol 202:497-505 (2006).

Nonetheless, protein infusion and, especially, gene delivery have the potential to provide diffuse distribution and long-lasting expression of a therapeutic protein in one surgical procedure. Adeno-associated type-2 (AAV2)-neurturin is a vector that has been genetically engineered to express and secrete the human gene for neurturin. Gasmi et al., Mol Ther 15:62-68 (2007). The AAV2 vector does not induce an inflammatory reaction, has been used safely in clinical trials, and provides long-lasting transgene expression. Bankiewicz et al., Mol Ther 14:564-570 (2006). An open-label, 12-month phase 1 trial of bilateral stereotactic intraputaminal injections of AAV2-neurturin in patients with advanced Parkinson's disease showed that the treatment was safe, well tolerated, and associated with benefits in motor functions. Marks Jr. et al., Lancet Neurol 7:400-408 (2008).

However, efficacy of the treatment in a Phase 2 trial did not fully fulfill the promise of the therapy, by not exceeding placebo responses to a statistically significant extent with respect to all primary goals. Thus, an urgent need still exists for a clinically efficacious gene therapy for Parkinson's disease.

### SUMMARY OF THE INVENTION

The present invention provides a clinically useful system and protocol for delivery of nerve growth factors into the mammalian brain. The invention is particularly useful in treating neurodegenerative conditions in primates, in whom nerve growth factors delivered according to the invention stimulate growth of neurons and recovery of neurological function.

In one aspect, the invention includes a method for delivery of a therapeutic nerve growth factor to targeted defective, diseased or damaged dopaminergic neurons in the brain of a human subject, such as those impaired in Parkinson's disease. The method includes directly delivering a nerve growth factor, a nerve growth factor encoding expression vector, or grafting a donor cell containing such an expression vector into the substantia nigra and, preferably, also into the striatum, for amelioration of the defect, disease or damage in response to the nerve growth factor. In one embodiment, striatal delivery is to at least one region of the putamen.

In various embodiments, the total unit dosage of nerve growth factor encoding expression vector delivered to the striatum is greater than the unit dosage of nerve growth factor encoding expression vector delivered into the substantia nigra. For example, the unit dosage delivered to the putamen may be up to 3, 4, 5, 6, 7, 8 or 9 times the unit dosage delivered to the substantia nigra.

In some embodiments, the disease is ameliorated by stimulation of repair of or activity in dopaminergic neurons. In related embodiments the disease is ameliorated by reversal of deficits in motor function associated with the Parkinson's disease.

In some embodiments, the nerve growth factor is a GDNF family molecule; for example, GDNF, neurturin, persephin or artemin.

In further embodiments, a recombinant expression vector used to deliver the nerve growth factor to cells for expression which is an AAV vector.

### BRIEF DESCRIPTION OF THE DRAWINGS

FIG. 1 provides a graphical illustration of an AAV2-neurturin recombinant expression vector. In the vector genome, AAV2 ITRs flank the NTN expression cassette, which consists of the CAG promoter, the pre-pro-NGF-NTN hybrid cDNA and the human growth hormone gene polyadenylation signal. The location of the canonical RXXR sequence derived from the NGF pro-domain and the cleavage site are shown.

### DETAILED DESCRIPTION OF THE INVENTION

General Caveats

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### Overview of the Invention

The present invention is based on the discovery that that delivery of a nerve growth factor deep in the neurocompromised brain, such as the substantia nigra of the Parkinson's disease brain, can provide enhancement of degenerating neurons. The invention provides an effective approach to overcome heretofore unrecognized deficiencies in axonal-transport along nigrastriatal neurons in advanced Parkinson's disease, which unexpectedly reduces the bioactivity of the delivered nerve growth factor by limiting the protein exposed to the cell body. This provides insight into targeting specific tissues to assure maximal benefit is achieved.

More particularly, during a Phase 2 clinical trial testing the efficacy and safety of AAV2-neurturin gene therapy in patients with advanced Parkinson's disease, two treated patients died from unrelated events, enabling analysis of post-treatment brain tissue in autopsy. Both cases had received four intraputaminal injections of boluses separated by 4mm in the dorso-ventral plane. Neurturin-immunolabeling was identified in all hemispheres examined. Quantitative volumetric analyses were performed independently using two different approaches and each revealed a mean coverage of the putamen of approximately 15% with some variation between hemispheres.

In areas with the densest neurturin immunolabeling, an increase in tyrosine hydroxylase (TH)-immunoreactive fibers could be observed. However, these fibers were always contained well within the sphere of neurturin signal. In contrast to all prior studies in animals, there was no clear evidence of retrograde transport of neurturin from the striatum to the nigra, nor evidence of TH induction in the nigra. These data call for a different approach to gene therapy of Parkinson's disease which is provided by the invention.

Before the present methods and methodologies are described, it is to be understood that this invention is not limited to the particular methods described as such methods may vary. It is also to be understood that the terminology used herein is for purposes of describing particular embodiments only, and is not intended to be limiting, since the scope of the present invention will be limited only in the appended claims.

Target Tissues.

The invention identifies and defines the required parameters of a method for successful regeneration of neurons in the brain with nerve growth factors, especially the neurons whose loss in the substantia nigra is associated with neurodegenerative conditions such as Parkinson's disease.

The first method parameter defined by the invention is selection of a suitable target tissue. A region of the brain is selected for its retained responsiveness to neurotrophic factors. In humans, CNS neurons which retain responsiveness to neurotrophic factors into adulthood include the cholinergic basal forebrain neurons, dopaminergic neurons of the substantia nigra, areas of the striatum, the putamen, the entorhinal cortical neurons, the thalamic neurons, the locus coeruleus neurons, the spinal sensory neurons and the spinal motor neurons. Loss of functionality in neurons of the substantia nigra is causatively associated with the onset of Parkinson's disease.

The substantia nigra is a relatively small deep brain structure, situated beneath the much larger striatum. Given the surgical risks involved in directly accessing the substantia nigra, delivery to it has been attempted via transport of expressed nerve growth factor from the striatum. However, in the Phase 2 studies described elsewhere above, delivery of an AAV-neurturin construct to the striatum unexpectedly failed to provide sufficient protein to the substantia nigra to achieve therapeutic results in humans comparable to those demonstrated using the same approach in non-human primate models of Parkinson's disease. It is believed that the nigrostriatal pathway may be more degenerated in human Parkinson's sufferers than previously understood.

As further defined in the Examples, delivery of a nerve growth factor according to the invention targets the substantia nigra (and preferably also the striatum). For striatal delivery, different regions of the striatum may be targeted, including for example, the putamen, globus pallidum and caudate nucleus.

Multiple areas of the brain may be targeted simultaneously, such as to both the putamen and substantia nigra. Additionally, multiple locations of the specific areas may be targeted, for example, 1, 2, 3, 4, 5, 6, 7, 8, 9 or 10 areas of the putamen and/or substantia nigra each along one or both hemispheres. In human patients, one or more injections per hemisphere or side to the substantia nigra and, preferably, one or more per hemisphere or side to the striatum are believed to be sufficient to achieve a therapeutic amelioration of the disease.

Dosing of the expression vector delivered to the striatum may also be greater than to the substantia nigra. For example, the total dose delivered to the striatum may be 1, 1.5, 2, 2.5, 3, 3.5, 4, 4.5, 5, 5.5, 6, 6.5, 7, 7.5, 8, 8.5, 9, 9.5 or 10 times the dose delivered to the substantia nigra in a single course of treatment. Most preferably, the total dose provided to the striatum is from 5 and 10 times the total dose provided to the substantia nigra.

As used in this disclosure, “unit dosage” refers generally to the concentration of nerve growth factor/ml of neurotrophic pharmaceutical composition prepared for use in the invention. For delivery via viral vectors (in vivo or ex vivo), the nerve growth factor concentration is defined by the number of viral particles/ml of neurotrophic composition. Optimally, for delivery of nerve growth factor using a viral expression vector, each unit dosage of nerve growth factor will comprise at least 2.5 μl of a neurotrophic composition, up to 25, 60, 100, 200, 300 μl or more as clinically indicated, wherein the composition includes a viral expression vector in a pharmaceutically acceptable fluid and provides from 10⁶ up to 10²⁰ expressing viral particles per ml of neurotrophic composition. For delivery of nerve growth factor protein, those of ordinary skill in the art will be readily able to convert dosing protocols along these lines to suitable unit doses.

Following the protocol defined by the invention, direct delivery of a nerve growth factor may be achieved by means familiar to those of skill in the art, including microinjection through a surgical incision (see, e.g., Capecchi, Cell, 22:479-488 (1980)), infusion, chemical complexation with a targeting molecule or co-precipitant (e.g., liposome, calcium), electroporation (see, e.g., Andreason and Evans, Biotechniques, 6:650-660 (1988)) and, for delivery of a nerve growth factor encoding expression vector composition, microparticle bombardment of the target tissue (Tang et al., Nature, 356:152-154 (1992)).

Those of skill in the art will appreciate that, for use in gene therapy especially, the direct delivery method employed by the invention obviates a limiting risk factor associated with gene therapy; to wit, the potential for transfection of non-targeted cells with the vector carrying the nerve growth factor encoding transgene. In the invention, delivery is direct and the delivery sites are chosen so diffusion of secreted nerve growth factor takes place over a controlled and predetermined region of the brain to optimize contact with targeted neurons, while minimizing contact with non-targeted cells. In addition, in primates and humans, viral vectors with an operable nerve growth factor encoding transgene have been shown to express human nerve growth factor after delivery to the brain for several years. As such, the invention provides a chronically available source for nerve growth factor in the brain.

### Materials for use in Practicing the Invention

Materials useful in the methods of the invention include in vivo compatible recombinant expression vectors, packaging cell lines, helper cell lines, synthetic in vivo gene therapy vectors, regulatable gene expression systems, encapsulation materials, pharmaceutically acceptable carriers and polynucleotides coding for nervous system growth factors of interest.

Nerve Growth Factors

Known nervous system growth factors include nerve growth factor (NGF), brain-derived neurotrophic factor (BDNF), nerve growth factor-3 (NT-3), nerve growth factor-4/5 (NT-4/5), nerve growth factor-6 (NT-6), ciliary neurotrophic factor (CNTF), glial cell line-derived neurotrophic factor (GDNF) and other members of the GDNF family of molecules (GDNF's naturally occurring analog, neurturin, as well as persephin and artemin), the fibroblast growth factor family (FGF's 1-15), leukemia inhibitory factor (LIF), certain members of the insulin-like growth factor family (e.g., IGF-1), the bone morphogenic proteins (BMPs), the immunophilins, the transforming growth factor (TGF) family of growth factors, the neuregulins, epidermal growth factor (EGF), platelet-derived growth factor (PDGF), and others.

The growth factors may be purified, synthesized or produced recombinantly. By “nerve growth factor” is meant growth factors of any origin which are substantially homologous to and which are biologically equivalent to the nerve growth factors referenced herein. Such substantially homologous growth factors may be native to any tissue or species and, similarly, biological activity can be characterized in any of a number of biological assay systems. For example, “nerve growth factors” can also include hybrid and modified forms of the molecules, including fusion proteins and fragments and hybrid and modified forms in which certain amino acids have been deleted or replaced and modifications such as where one or more amino acids have been changed to a modified amino acid or unusual amino acid and modifications such as glycosolations so long as the hybrid or modified form retains the biological activity of the subject nerve growth factor. By retaining the biological activity, it is meant that neuronal repair is achieved or activity promoted, although not necessarily at the same level of potency as that of the nerve growth factor in isolated and purified form or that which has been recombinantly produced.

Reference to pre-pro sequences of nerve growth factors herein is intended to be construed to include pre-pro growth factors containing a pre- or leader or signal sequence region, a pro-sequence region and mature protein. The nucleotide sequences of pre- and/or pro-regions can also be used to construct chimeric genes with the coding sequences of other growth factors or proteins and, similarly, chimeric genes can be constructed from the coding sequence of the subject nerve growth factor coupled to sequences encoding pre- and/or pro-regions from genes for other growth factors or proteins (Booth et al., Gene 146:303-8 (1994); Ibanez, Gene 146:303-8 (1994); Storici et al., FEBS Letters 337:303-7 (1994); Sha et al, J Cell Biol 114:827-839 (1991), which are incorporated by reference). Such chimeric proteins can exhibit altered production or expression of the active protein species.

Particularly exemplary nerve growth factors for use with the invention include the GDNF family of GDNF, neurturin (NRTN), persephin and artemin (e.g., for treatment of Parkinson's disease). The coding sequences for these nerve growth factors are well known to, or readily identifiable by, those of ordinary skill in the art and need not be repeated here.

For the GDNF family, for example, reference may be made to the nucleotide sequences set forth in:
www.ncbi.nlm.nih.gov/gene/2668 (Gene ID 2668, human GDNF),
www.ncbi.nlm.nih.gov/gene/4902 (Gene ID 4902, human neurturin—see also, U.S. Pat. No. 6,090,778),
www.ncbi.nlm.nih.gov/gene/5623 (Gene ID 5623; human persephin), and
www.ncbi.nlm.nih.gov/gene/9048 (Gene ID 9048; human artemin),
and other sources.

For the GDNF family, for example, reference may be made to the polypeptide sequences set forth in:
www.ncbi.nlm.nih.gov/protein/CAG46721.1 (Accession No. CAG46721; human GDNF);
www.ncbi.nlm.nih.gov/protein/EAW69140.1 (Accession No. EAW69140; human neurturin—see also, U.S. Pat. No. 6,090,778);
www.ncbi.nlm.nih.gov/protein/AAC39640.1 (Accession No. AAC39640; human persephin),
www.ncbi.nlm.nih.gov/protein/AAD13109.1 (Accession No. AAD13109.1; human artemin),
and other sources.

Human nerve growth factors are preferred for use in therapy of human disease according to the invention due to their relatively low immunogenicity as compared to allogenic growth factors. However, other nerve growth factors are known which may also be suitable for use in the invention with adequate testing of the kind described herein.

Recombinant Expression Vectors

The strategy for transferring genes into target cells in vivo includes the following basic steps: (1) selection of an appropriate transgene or transgenes whose expression is correlated with CNS disease or dysfunction; (2) selection and development of suitable and efficient vectors for gene transfer; (3) demonstration that in vivo transduction of target cells and transgene expression occurs stably and efficiently; (4) demonstration that the in vivo gene therapy procedure causes no serious deleterious effects; and (5) demonstration of a desired phenotypic effect in the host animal.

Although other vectors may be used, preferred vectors for use in the methods of the present invention are viral and non-viral vectors, such as DNA vectors (e.g, adeno-associated virus (AAV) and adenovirus, especially the former). The vector selected should meet the following criteria: 1) the vector must be able to infect targeted cells and thus viral vectors having an appropriate host range must be selected; 2) the transferred gene should be capable of persisting and being expressed in a cell for an extended period of time (without causing cell death) for stable maintenance and expression in the cell; and 3) the vector should do little, if any, damage to target cells.

Because adult mammalian brain cells are non-dividing, the recombinant expression vector chosen must be able to transfect and be expressed in non-dividing cells. Vectors known to have this capability include DNA viruses such as adenoviruses, adeno-associated virus (AAV), and certain RNA viruses such as HIV-based lentiviruses, feline immunodeficiency virus (FIV) and equine immunodeficiency virus (EIV). Other vectors with this capability include herpes simplex virus (HSV).

Construction of vectors for recombinant expression of nervous system growth factors for use in the invention may be accomplished using conventional techniques which do not require detailed explanation to one of ordinary skill in the art. A specific protocol for construction of an AAV vector useful in the invention is illustrated in the Examples. Use of the AAV2 serotype is exemplified; however, other known AAV serotypes might be employed. For further review regarding general techniques for vector construction, those of ordinary skill may wish to consult Maniatis et al., in Molecular Cloning: A Laboratory Manual, Cold Spring Harbor Laboratory, (NY 1982).

Promoter and enhancer regions of a number of viral and non-viral promoters have also been described (e.g., as to non-viral promoters, Schmidt et al., Nature 314:285 (1985); Rossi and de Crombrugghe, Proc. Natl. Acad Sci. USA 84:5590-5594 (1987)). Methods for maintaining and increasing expression of transgenes in quiescent cells include the use of promoters including collagen type I (1 and 2) (Prockop and Kivirikko, N. Eng. J. Med. 311:376 (1984); Smith and Niles, Biochem. 19:1820 (1980); de Wet et al., J. Biol. Chem., 258:14385 (1983)), SV40, chicken β-actin, and LTR promoters.

Transgene expression may also be increased for long term stable expression using cytokines to modulate promoter activity. For example, transforming growth factor (TGF), interleukin (IL)-1, and interferon (INF) down regulate the expression of transgenes driven by various promoters such as LTR. Tumor necrosis factor (TNF) and TGF1 up regulate, and may be used to control, expression of transgenes driven by a promoter. Other cytokines that may prove useful include basic fibroblast growth factor (bFGF) and epidermal growth factor (EGF).

Collagen promoter with the collagen enhancer sequence (Coll(E)) can also be used to increase transgene expression by suppressing further any immune response to the vector which may be generated in a treated brain notwithstanding its immune-protected status. In addition, anti-inflammatory agents including steroids, for example dexamethasone, may be administered to the treated host immediately after vector composition delivery and continued, preferably, until any cytokine-mediated inflammatory response subsides. An immunosuppression agent such as cyclosporin may also be administered to reduce the production of interferons, which downregulates LTR promoter and Coll(E) promoter-enhancer, and reduces transgene expression.

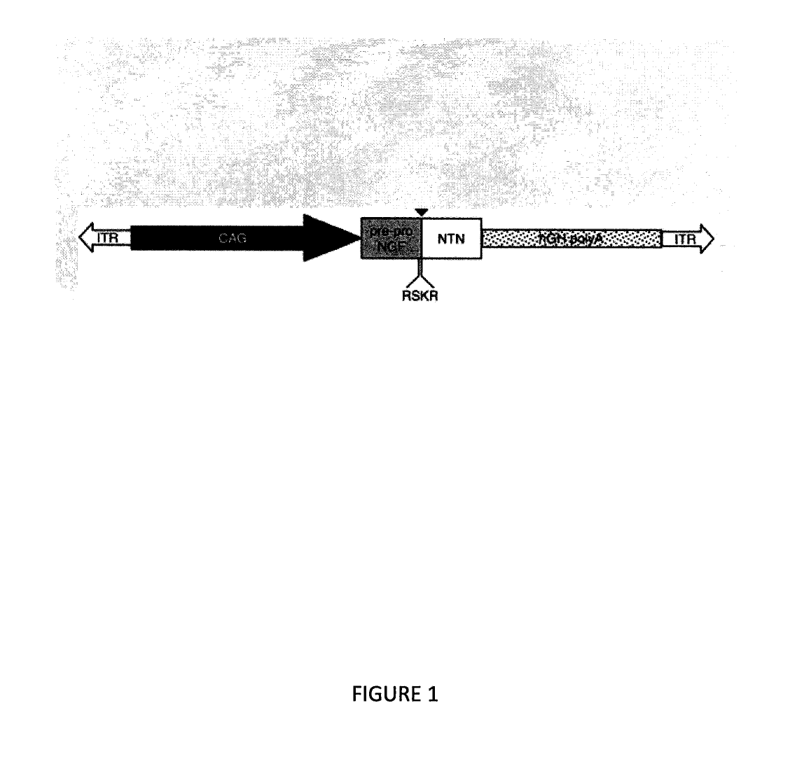

Construction of an AAV2 viral vector containing a human neurturin cDNA sequence is described in FIG. 1 and in Gasmi et al., Mol Ther 15:62-68 (2007), which disclosure is incorporated herein by this reference. A pre-pro form of neurturin is cleaved to form the mature protein and the human pre-pro form containing the pre-pro region. To enhance secretion of the mature neurturin molecule, signal peptide sequences other than pre-pro neurturin may be employed as described in U.S. Pat. No. 6,090,778; e.g., one drawn from NGF (Gasmi, et al., ibid.).

Donor Cells

While the present disclosure focuses on the preferred in vivo delivery methods, those of ordinary skill in the art will recognize that host cells, such as fibroblasts and stem cells (including, without limitation, embryonic stem cells and adult induced pluripotent or totipotent stem cells), may also be utilized for ex vivo delivery of nerve growth factor encoding expression vectors to the brain. Preparation of donor cells containing a nerve growth factor transgene encoding expression vector is described in detail in commonly assigned U.S. Pat. No. 5,650,148, the contents of which are incorporated herein. The preparation is carried out by modifying donor cells by introduction of a vector containing a transgene or transgenes encoding a nerve growth factor protein, which cells are in turn grafted onto the target tissue.

The preparation of donor cells is known to those of ordinary skill in the art. Briefly, the strategy for transferring genes into donor cells in vitro includes the following basic steps: (1) selection of an appropriate transgene or transgenes whose expression is correlated with CNS disease or dysfunction; (2) selection and development of suitable and efficient vectors for gene transfer; (3) preparation of donor cells (e.g., from primary cultures or from established cell lines); (4) demonstration that the donor implanted cells expressing the new function are viable and can express the transgene products(s) stably and efficiently; (5) demonstration that the transplantation causes no serious deleterious effects; and (6) demonstration of a desired phenotypic effect in the host animal.

Pharmaceutical Preparations

Direct in vivo delivery of a nerve growth factor encoding expression vector is preferred for use in the invention. To that end, nerve growth factor encoding expression vectors may be placed into a pharmaceutically acceptable suspension, solution or emulsion. Similar carriers may be employed for delivery of nerve growth factor protein.

More specifically, pharmaceutically acceptable carriers may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

The pharmaceutical compositions of the present invention can be administered by any suitable route known in the art including for example intravenous, subcutaneous, intramuscular, transdermal, intrathecal or intracerebral. Administration can be either rapid as by injection or over a period of time as by slow infusion or administration of slow release formulation. For treating tissues in the central nervous system, administration can be by injection or infusion into the cerebrospinal fluid (CSF). When it is intended that the nerve growth factors be administered to cells in the central nervous system, administration can be with one or more agents capable of promoting penetration of the molecule across the blood-brain barrier.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion into the cerebrospinal fluid by continuous or periodic infusion. Preservatives and other additives may also be present such as, for example, antimicrobials, antioxidants, chelating agents, and inert gases and the like. Further, a composition of nerve growth factor transgenes may be lyophilized using means well known in the art, for subsequent reconstitution and use according to the invention.

Dose administration can be repeated depending upon the pharmacokinetic parameters of the dosage formulation and the route of administration used. The specific dose is calculated according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macro molecules. RNA, DNA and intact virions can be encapsulated within the aqueous interior and be delivered to cells in a biologically active form (Fraley, et al., Trends Biochem. Sci., 6:77, 1981).

In order for a liposome to be an efficient gene transfer vehicle, the following characteristics should be present: (1) encapsulation of the genes encoding the antisense polynucleotides at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted gene delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

Animal Models and Clinical Evaluation

In non-human primate subjects, the process of aging simulates the neurological changes in the brain experienced in aging humans. A non-aged animal model that models Parkinson's disease with a high degree of integrity is 1-methyl-4-phenyl-1,2,3,6-tetrahydropyridine (MPTP) treated monkeys (see, e.g., Kordower et al., Exp. Neurology, 160:1-16 (1999)). Such treatment results in extensive degeneration of dopaminergic neurons in the substantia nigra, with concomitant behavioral modification and motor deficits (Example 2). Data demonstrating the use and efficacy of the method of the invention in aged non-human primates as well as MPTP treated animals has been previously demonstrated. Further, as discussed in the Examples, data demonstrating the use of the method of the invention in humans has also been demonstrated.

Clinical evaluation and monitoring of treatment can be performed using the in vivo imaging techniques described above as well as through biopsy and histological analysis of treated tissue. In the latter respect, neuronal numbers can be quantified in a tissue sample with respect to, for example, TH immunoreactivity.

Clinical evaluation and monitoring of amelioration of symptoms of disease may be assessed and observed at various time points post-treatment. In various embodiments amelioration is observable at or after 30 days, 60 days, 90 days, 180 days, 12 months, 18 months, 24 months, 48 months, 72 months, and longer post-treatment.

Patients may be assessed with the URPDRS in the practically-defined off state (around 12 h after the last dose of antiparkinsonian drug) and in the best on state (best response to morning dose of antiparkinsonian drug). Fahn et al., Recent Developments in Parkinson's disease, Macmillan Healthcare Information, Florham Park, N.J. 2:153-163 (1987). The motor subscale (part 3) of the UPDRS may also be done in the practically-defined off state at each visit after 12 months. Home diary assessment of motor state, timed motor tests, the dyskinesia rating scale, and the clinical global impression assessment may be done periodically; e.g., at baseline, 6 months, and at each visit thereafter. Quality of life may be assessed with the Parkinson's disease questionnaire (PDQ)-39 (Jenkinson et al., Age Ageing 26:353-357 (1997)) and short form (SF)-36 (Ware et al., Med Care 30:473-483 (1992)) periodically; e.g., at baseline and subsequently thereafter.

The invention having been fully described, examples illustrating its practice are set forth below. These examples should not, however, be considered to limit the scope of the invention, which is defined by the appended claims.

### EXAMPLE 1

### Bioactivity of AAV2-Neurturin Gene Therapy (AAV-Neurturin): Differences Between Parkinson's Disease and Nonhuman Primate Brains

A Phase 1 trial in 12 moderately advanced Parkinson's disease patients identified no safety issues, while suggesting possible improvement on several measures of motor function. Marks et al., Lancet Neurol 7:400-408 (2008). As noted elsewhere above, a subsequent double-blind-controlled Phase 2 trial in 58 subjects further supported the safety of AAV-neurturin, but failed to discern any benefit compared to sham surgery on the primary endpoint (UPDRS-motor-“off” at 12-months). However, several secondary endpoints suggested modest clinical benefit, while no measurement favored sham-control. Moreover, a protocol-prescribed analysis of all data from patients whose treatment remained blinded at 15 to 18 months (n=30) suggested significant benefit with AAV-neurturin (AAV2-NTRN) on the primary and secondary endpoints with no measurement favoring sham.

Two Parkinson's disease patients in the Phase 2 trial died from non-AAV2-NRTN-related events, and their brains were examined histologically. The effects of delivering AAV2-NRTN to the putamen in these patients, comparing the expression of NRTN in putamen and nigra, and TH-induction in nigrostriatal neurons, to that following the same treatment in young, aged, and Parkinsonian monkeys are detailed below.

Vector Design and Construction

AAV-neurturin was utilized which is an AAV2 vector genetically engineered to express only human NRTN. Gasmi et al., Mol Ther 15:62-68 (2007).

Parkinson's Autopsy Cases

Patients who previously met entry criteria and signed an IRB-approved informed consent received AAV-neurturin (5.4×10¹¹ vg), distributed via four separate needle tracts per hemisphere and two deposits per tract. One subject (#1802) was a 59-year-old man who had been diagnosed 10.2 years earlier and died from a pulmonary embolism on Day 90, post-AAV-neurturin treatment. The second subject (#1904) was a 73-year-old man who had been diagnosed 9.5 years earlier and suffered a fatal myocardial infarction on Day 47 post-AAV-neurturin. Subject #1904 had baseline UPDRS motor off/on scores of 34/21 and a self-reported diary off time of 1.33 hr/day. Subject #1802 had baseline UPDRS motor off/on scores of 51/34 and a self-reported diary off time of 4.7 hr/day.

Brains were fixed following postmortem intervals of 6 and 13 hours, respectively, with a modified Zamboni's solution and sectioned at 40 μm. Only one hemisphere was available from the second subject. Brains were stained for NRTN and tyrosine hydroxylase (TH), using non-Parkinson's disease aged humans as positive controls. Both brains demonstrated typical pathological features of Parkinson's disease, with marked loss of cells in the substantia nigra, coupled with multiple α-synuclein-stained Lewy bodies.

Nonhuman Primate Cases

Ten Rhesus monkeys (Macaca mulatto) were administered intraputaminal doses of AAV2-NRTN within the range administered to the Parkinson's disease patients, based on relative striatal volume, except for two young monkeys who intentionally received a substantially lower dose (˜4% of human dose by striatal volume) and their brains evaluated only 28 days later. All housing and experimentation was IACUC approved. Kordower et al., Ann Neurol 60:706-715 (2006); Herzog et al., Mov Disord 22:1124-1132 (2007); Herzog et al., Mol Ther 16:1737-1744 (2008); and Herzog et al., Neurosurgery 64:602-612 (2009). Following AAV-neurturin administration, monkeys were anesthetized, perfused transcardially with 0.9% saline, followed by a modified Zamboni's fixative and the brains sectioned. Some of these monkeys were part of previous publications, though the specific data presented here were not previously published. Kordower et al., Ann Neurol 60:706-715 (2006); Herzog et al., Mov Disord 22:1124-1132 (2007); Herzog et al., Mol Ther 16:1737-1744 (2008).

Two young monkeys (˜5 years old) were administered 0.5×10¹¹ vg of AAV-neurturin per hemisphere in two deposits in a total volume of 25 μL within the striatum and euthanized 1 month later, and an additional young monkey received 1.0×10¹¹ vg per hemisphere of AAV-neurturin and was euthanized three months later.

Three aged monkeys (22-25 years old) were administered 3×10¹¹ vg of AAV-neurturin, unilaterally, via five deposits (30 μL each) distributed throughout the striatum and euthanized 8 months later.

Five monkeys (age range: 6-10 years) received unilateral, intracarotid infusions of MPTP resulting in motor dysfunction. They were then administered 1.5×10¹¹ vg of AAV-neurturin 4 days later, via five deposits (15 μL each) distributed throughout the striatum, as well as a single, 10 μL injection into the substantia nigra (0.2×10¹¹ vg dose) and euthanized 10.5 months later.

Immunohistochemical Analysis of Neurturin and Tyrosine Hydroxylase

Immunoperoxidase labeling was used to visualize NRTN and TH within the human and nonhuman primate striatum and substantia nigra as described in Kordower et al., Ann Neurol., 60:706-715 (2006). A stereologic sampling method, combined computer-assisted imaging software, and the Cavalieri method was used to quantify volume of NRTN expression in the primate striatum. For the human cases, two different methods were independently employed to compute volume of NRTN expression in putamen. One method employed volumetric analyses based on stereological sampling of 6 to 11 sections throughout each putamen. The second method sampled all sections found to contain putamen (19-29 sections per each case).

The percent of NRTN expressed within each targeted structure was then calculated based on the volumes of the entire target, using values from in-house human and primate histological sections and MRI scans, as well as published values to provide an estimate of nonhuman primate caudate/putamen of ˜1200 mm³/hemisphere and human putamen of ˜4000 mm³/hemisphere).

Results

NRTN Expression

For the two Parkinson's disease cases, neurturin expression was quantified in the three available hemispheres 7 weeks or 3+ months post-AAV2-NRTN treatment following death from unrelated causes. NRTN-immunoreactivity was seen in all hemispheres, restricted to the targeted putamen. Two independent, blinded analyses conservatively estimated NRTN protein covered ˜15% of the entire putamen by volume. The dosing paradigm employed for AAV-neurturin was intended to distribute the AAV2-NRTN as widely as possible throughout the putamen, while limiting spread to surrounding sites to reduce potential side effects. Kordower et al. Ann Neurol 46:419-424 (1999); Nutt et al., Neurology 60:69-73 (2003); and Eriksdotter et al., Dement Geriatr Cogn Disord 9:246-257 (1998). Detectable NRTN protein was seen in 93%, 58%, and 80% of all putaminal sections analyzed, in each of the three hemispheres studied.

In contrast to the strong NRTN expression in the putamen (i.e., terminal field of the dopamine nigral neurons), very little NRTN staining was seen in the substantia nigra (i.e., neuronal cell bodies of these same neurons) despite appreciable, surviving dopaminergic, melanin-positive neurons.

Two young monkeys were administered a particularly low dose of AAV-neurturin (less than 4% of the human Parkinson's disease dose, by relative volume of each targeted structure) and euthanized only 1 month later, to provide a conservative estimate of the early-onset bioactivity of AAV-neurturin with low NRTN expression levels. The volume of striatal NRTN expression in these two monkeys was estimated to be only 5.6% and 1.8%. Despite this low level of striatal NRTN expression, and in contrast to the Parkinson's disease cases, NRTN retrograde labeling was easily seen within substantia nigra perikarya and anterogradely transported NRTN⁺ fibers were seen coursing within the globus pallidus and substantia nigra pars reticulata.

For the three aged monkeys administered AAV2-NRTN eight months earlier, NRTN was estimated to cover 4, 19, and 25% (mean 16%) of the entire striatum, by volume. Despite the variation in striatal NRTN coverage, NRTN was consistently observed in the substantia nigra, in contradistinction to the Parkinson's disease tissue.

For the five MPTP-treated monkeys (administered AAV2-NRTN 4 days following MPTP treatment euthanized 10 months later), a mean of ˜13% of the entire putamen by volume stained for NRTN (8%, 8%, 13%, 15%, and 23%, respectively). Similar to other primate studies, extensive evidence for retrogradely and anterogradely transported NRTN within the substantia nigra pars compacta and reticulata, respectively, was observed.

Tyrosine Hydroxylase Immunohistochemistry

Examination of the human Parkinson's disease autopsy tissue found only scant evidence for TH induction following AAV-neurturin. The clearest evidence for TH induction was observed in the targeted striatum, well within the boundaries of some of the most intense sites of NRTN staining. This signal, reflecting only sparse TH-positive fibers, was observed on average in 50% of the NRTN-positive putaminal sites (i.e., 0%, 62%, and 80%, respectively). No evidence for TH induction was found in the substantia nigra of the Parkinson's disease brains, despite the presence of numerous melanin-containing, TH+ dopaminergic neurons.

In contrast to the sparse TH-induction signal in the Parkinson's disease cases, TH-induction following AAV2-NRTN treatment in monkeys was consistently observed in nigrostriatal neurons, was generally robust and typically mirrored the extent and intensity of NRTN expression in the striatum and the nigra. This was evident even at 28 days postdosing, with substantially lower doses, and less NRTN expression, than in the Parkinson's disease cases. Finally, in further contrast to the TH response in Parkinson's disease brain, the area of TH induction in monkey striatum consistently exceeded that of NRTN staining in adjacent sections.

By the above data, it is shown that gene therapy can produce targeted expression of a potentially potent therapeutic protein deep within the brains of neurodegenerative patients. Administration of AAV2-NRTN (AAV-neurturin) to the putamen in the Parkinson's disease brain resulted in clear expression of NRTN in the targeted putamen, conservatively estimated to cover ˜15% of the structure by volume. While it is not known how much coverage is required for clinical benefit in Parkinson's disease patients, this amount is clearly within the range that provided biological benefit in several nonhuman primate models and the accumulated animal and human safety data now provide the justification for responsibly expanding that coverage further. Kordower et al., Ann Neurol 60:706-715 (2006); Herzog et al., Mov Disord 22:1124-1132 (2007); Herzog et al., Mol Ther 16:1737-1744 (2008); and Herzog et al., Neurosurgery 64:602-612 (2009).

However, in contrast to all prior animal studies, NRTN expression in the Parkinson's disease putamen did not result in labeling of the neuronal cell bodies in the substantia nigra, despite putaminal coverage more than sufficient to produce this response in young, aged, and MPTP-parkinsonian monkeys. This distinction suggests a profound difference in the status and function of nigrostriatal neurons in advanced Parkinson's disease versus typical animal models used for Parkinson's disease translational research. As illustrated in the following Examples, the invention has taken this difference into account.

Of equal interest is the modest AAV2-NRTN-mediated TH-induction seen in the putamen following AAV2-NRTN administration and subsequent NRTN expression (TH is a major enzyme for dopamine synthesis and a surrogate for functional enhancement of degenerating dopamine neurons). The robust TH signal in nonhuman primates in response to AAV-NRTN is in marked contrast to the limited signal in Parkinson's disease in a number of important ways, including: (1) the intensity of TH signal was far less in Parkinson's disease, (2) it occurred with less frequency and reliability, and (3) it occurred within a much smaller portion of the putamen, well within the region of NRTN expression.

Conventional wisdom, based on considerable animal research with neurotrophic factors (primarily GDNF) in degenerating nigrostriatal neurons, argued that targeting the terminal fields of these neurons (i.e., the striatum) is both necessary and sufficient to gain optimal neurotrophic benefit (and that targeting the substantia nigra is unnecessary or even counter-productive. Kirik et al., Nat Neurosci 7:105-110 (2004); Bjorklund et al., Neurobiol Dis 4:186-200 (1997); and Kirik et al., J Neurosci 20:4686-4700 (2000). This perspective was further supported by seminal research with neurotrophic factors demonstrating that they most often function by being taken-up by the neuron's terminals and retrogradely transported to their cell bodies to induce trophic effects. Mufson et al., Prog Neurobiol 57:451-484 (1999) and Lindsay et al., Trends Neurosci 17:182-190 (1994). Indeed, delivering GDNF or NRTN to the terminal field in the striatum has consistently been shown to be sufficient to elevate GDNF and NRTN levels in both the axon terminals as well as the cell bodies in the nigra via retrograde transport. Salvatore et al., Exp Neurol 202:495-505 (2006); Ai et al., J Comp Neurol 461:250-261 (2003); Su et al., Human Gene Ther 20:1627-1640 (2009); and Tomac et al., Nature 373:335-339 (1995).

However, the data herein that this does not occur in a similar fashion in advanced Parkinson's disease, revealed by the paucity of NRTN-positive perikarya in the nigra, despite clear NRTN in the putamen and sufficient dopamine neurons in the substantia nigra. The evidence for a similarly weak NRTN signal in the human substantia nigra following putaminal delivery, strategies assuring greater neurotrophic exposure to degenerating perikarya in the substantia nigra would provide a more rapid and robust neurotrophic response and thus more meaningful clinical benefit.

Contrary data showing injections of GDNF into the nigra, in addition to the striatum, are without benefit and may be harmful should be interpreted with care. Kirik et al., J Neurosci 20:4686-4700 (2000). These data merely show that when sufficient nigral GDNF exists from retrograde transport of striatal GDNF, the additional targeting of the nigra is unnecessary. However, when the degeneration in nigrastriatal pathways prevent retrograde transport of NRTN from the striatum to the substantia nigra in humans with advanced Parkinson's disease, the basis for a conclusion that targeting of the nigra is unnecessary breaks down.

### EXAMPLE II

### Multicenter, Randomized, Double-Blind, Sham Surgery-Controlled Study of Intraputaminal AAV2-Neurtin (AAV-Neurturin) for Advanced Parkinson's Disease

AAV2 vector was genetically engineered to express only human neurturin (NRTN) as discussed in Example I and Gasmi et al., Mol Ther 15:62-68 (2007). It provides targeted and sustained delivery of neurturin (NRTN) to cells of the brain.

To conduct the multicentre, double-blind, sham-surgery controlled trial using AAV2-NRTN patients were randomly assigned (2:1) by a central, computer generated, randomization code to receive either AAV2-neurturin (5.4×10¹¹ vector genomes) injected bilaterally into the putamen or sham surgery. All patients and study personnel with the exception of the neurosurgical team were masked to treatment assignment. The primary endpoint was change from baseline to 12 months in the motor subscore of the unified Parkinson's disease rating scale in the practically-defined off state. All randomly assigned patients who had at least one assessment after baseline were included in the primary analyses.

Between December, 2006 and November, 2008, 58 patients from nine sites in the USA participated in the trial. There was no significant difference in the primary endpoint in patients treated with AAV2-neurturin compared with control individuals (difference −0.31 [SE 2.63], 95% CI −5.58 to 4.97; p=0.91). Intraputaminal AAV2-neurturin was therefore not superior to sham surgery when functionally assessed using the UPDRS motor score at 12 months.

### EXAMPLE III

### Delivery of AAV-Neurturin to the Substantia Nigra and Putamen

AAV2-neurturin is being utilized in a Phase 2b multi-center, sham-surgery, double-blinded controlled trial in advanced Parkinson's disease initiated in October 2010. Advanced patients can be expected to have greater degeneration of their nigrastriatal transport pathways than patients in earlier stages of the disease, in which the invention is therefore expected to readily demonstrate efficacy. As of this filing, approximately 20 percent of the 52 subjects have undergone either CERE-120 administration or sham surgery, with many others enrolled and awaiting surgery. The protocol employs the present invention along the parameters outlined below.

Stereotactic surgery is done with neuroimaging to plan injection trajectories. Patients are anaesthetized with deep propofol sedation. For patients assigned to active treatment, a gene transfer procedure is done with AAV2 as a vector to deliver DNA-encoding neurturin to the putamen. AAV2-neurturin in a total brain dose of 5.4×10¹¹ vector genomes is administered bilaterally. In these patients, the substantia nigra is directly targeted at two injection sites (reached through burr holes) per side, with a higher dose delivered into the putamen according to the invention, with putaminal delivery being made to three injection sites per side.

This Phase 2b trial was initiated following the successful dosing of six patients in a Phase 1 safety trial that evaluated, for the first time, the feasibility and safety of targeting the substantia nigra with AAV-neurturin, as well as administering a larger dose than had been tested previously. The Phase 1 safety database currently reflects follow-up periods ranging from seven to 13 months per patient, and shows no serious adverse events (SAEs) in any of the six subjects dosed, including no effect on weight. All patients were discharged from the hospital within 48 hours of surgery, as planned. Consistent with the safety profile observed in the Phase 1 trial, no AAV-neurturin-related serious adverse events have been observed in the ongoing Phase 2 trial.

Patients are assessed at baseline and months 1, 3, 6, 9, and 12 after surgery and every 3 months thereafter until the final patient enrolled completes a 15-month evaluation, as outlined with respect to human clinical protocols elsewhere above. Amelioration of neurodegenerative deficits are expected to be observable as early as 3 months following treatment. Benefits emerging after 12 months in patients who underwent putaminal treatment in the prior clinical trial suggested that tracking after the one year time point may also evidence further amplification of the neurturin signal in the striatum and cell bodies of the substantia nigra; e.g., at 18, 24, 48 or 72 months following treatment, and potentially thereafter.

Although the invention has been described with reference to the above examples, it will be understood that modifications and variations are encompassed within the spirit and scope of the invention. Accordingly, the invention is limited only by the following claims.

What is claimed is:

1. A method for delivery of a therapeutic nerve growth factor to targeted defective, diseased or damaged dopaminergic neurons in the brain of a human subject, the method comprising directly delivering a nerve growth factor encoding expression vector into the substantia nigra and into the striatum, for amelioration of the defect, disease or damage in response to expressed nerve growth factor.

2. The method according to claim 1, wherein the direct delivery to the striatum is to at least one region of the putamen.

3. The method according to claim 1, wherein the total unit dosage of nerve growth factor encoding expression vector delivered to the striatum is greater than the unit dosage of nerve growth factor encoding expression vector delivered into the substantia nigra.

4. The method according to claim 3, wherein the unit dosage delivered to the putamen is up to 10 times the unit dosage delivered to the substantia nigra.

5. The method according to claim 1, wherein the disease is ameliorated by stimulation of repair or activity in dopaminergic neurons.

6. The method according to claim 1, wherein the disease is ameliorated by reversal of deficits in motor function associated with the Parkinson's disease.

7. The method according to claim 1, wherein the nerve growth factor is from the GDNF family selected from the group of molecules consisting of GDNF, neurturin, persephin and artemin.

8. The method according to claim 7, wherein the nerve growth factor is neurturin.

9. The method according to claim 1, wherein the expression vector is an adeno-associated viral vector.

10. The method according to claim 9, wherein the AAV vector is an AAV serotype 2 vector.

11. The method according to claim 1, wherein the delivery is performed with a pump.

12. The method according to claim 1, wherein retrograde transport to the substantia nigra is impaired in the subject.

13. The method according to claim 4, wherein the unit dosage delivered to the putamen is 4 times the unit dosage delivered to the substantia nigra.

14. The method according to claim 6, wherein the subject has advanced Parkinson's disease.

15. The method according to claim 6, wherein the subject has early Parkinson's disease.

16. The method according to claim 6, wherein the amelioration is observable at or after 1 month post-treatment.

17. The method according to claim 6, wherein the amelioration is observable at or after 12 months post-treatment.

18. The method according to claim 6, wherein the amelioration is observable at or after 18 months post-treatment.

19. The method according to claim 6, wherein the amelioration is observable at or after 24 to 48 months post-treatment.

20. The method according to claim 1, wherein the nerve growth factor is expressed in cell bodies of the substantia nigra.

21. The method according to claim 1, wherein TH upregulation occurs in the striatum.

22. The method according to claim 1, wherein TH upregulation occurs in the substantia nigra.

23. The method according to claim 1, wherein delivery in the striatum is to at least 1 site per side of the brain.

24. The method according to claim 1, wherein delivery to the substantia nigra is to at least 1 site per side of the brain.

25. A method for delivery of a therapeutic nerve growth factor to targeted defective, diseased or damaged dopaminergic neurons in the brain of a human subject, the method comprising directly delivering the nerve growth factor into the substantia nigra and into the striatum, for amelioration of the defect, disease or damage in response to expressed nerve growth factor.

26. A method for delivery of a therapeutic nerve growth factor to targeted defective, diseased or damaged dopaminergic neurons in the brain of a human subject, the method comprising directly delivering a nerve growth factor encoding expression vector into the substantia nigra, for amelioration of the defect, disease or damage in response to expressed nerve growth factor.

27. A method for delivery of a therapeutic nerve growth factor to targeted defective, diseased or damaged dopaminergic neurons in the brain of a human subject, the method comprising directly delivering the nerve growth factor into the substantia nigra, for amelioration of the defect, disease or damage in response to expressed nerve growth factor.

28. A method for delivery of a therapeutic nerve growth factor to targeted defective, diseased or damaged dopaminergic neurons in the brain of a human subject, the method comprising directly grafting a nerve growth factor contained in a donor cell into the substantia nigra and into the striatum, for amelioration of the defect, disease or damage in response to expressed nerve growth factor.

29. A method for delivery of a therapeutic nerve growth factor to targeted defective, diseased or damaged dopaminergic neurons in the brain of a human subject, the method comprising directly grafting a nerve growth factor contained in a donor cell into the substantia nigra and into the striatum, for amelioration of the defect, disease or damage in response to expressed nerve growth factor.
